(19) United States
(12) Patent Application Publication
(10) Pub. No.: US 2018/0104008 A1
(43) Pub. Date: Apr. 19, 2018

(54) COMMUNICATING LOCALIZATION MARKERS

(71) Applicant: COVIDIEN LP, MANSFIELD, MA (US)

(72) Inventor: WILLIAM J. DICKHANS, LONGMONT, CO (US)

(21) Appl. No.: 15/293,939

(22) Filed: Oct. 14, 2016

(51) Int. Cl. A61B 34/20; A61B 90/98; A61B 90/00; A61B 5/06

(52) U.S. Cl. CPC A61B 34/20; A61B 90/98; A61B 2034/2051; A61B 5/064; A61B 5/062; A61B 90/39

(57) ABSTRACT

A surgical system including a fiducial, a surgical instrument, and an indicator is provided. The fiducial is configured for insertion into target tissue and has an RFID tag disposed therein. The surgical instrument is capable of receiving information from the RFID tag when it is placed in proximity to the fiducial and is configured to perform a surgical function. The indicator is coupled to the surgical instrument and is configured to indicate when the surgical instrument is in proximity to the fiducial. A method of performing a surgical procedure is also provided.

### BACKGROUND

### Technical Field

The present disclosure relates to systems and methods for tissue localization, and more particularly, to a system and method incorporating communicating fiducials for use in surgical procedures.

### Description of Related Art

Modern technology has had a profound impact on surgical techniques. As a result, the quality of patient care has improved through the use of techniques such as minimally invasive therapies, radiofrequency ablation, cryosurgery, photodynamic therapy, brachytherapy, and microwave ablation. When performing these or similar procedures, the surgeon must be able to accurately determine the position of the surgical instrument relative to the tissue undergoing treatment. Typically, during an open procedure, the surgeon would have direct line of sight to the surgical instrument. However, with the increasing reliance upon minimally invasive techniques, such as endoscopic, thoracoscopic, laparoscopic, etc., it is often impossible for the surgeon to be able to identify the position of the surgical instrument within the patient.

With the advent of imaging modalities such as Computed Tomography (CT), Magnetic Resonance Imaging (MRI), Positron Emission Tomography (PET), ultrasound, X-ray imaging, Flouroscopy, etc., surgeons have been able to more accurately identify lesions, and therefore, more accurately treat lesions within a patient while minimizing trauma to surrounding tissues.

To further improve the accuracy and efficacy of surgical procedures, fiducials may be implanted within the lesion or in the tissue identified to be removed. These fiducials, in many cases formed from gold, are implanted within the lesion through the use of preloaded fiducial needles or aspiration needles and are identified using intraoperative imaging such as CT scans or fluoroscopy. Identification of these fiducials allows a surgeon to more accurately remove the lesion while minimizing the removal of excess tissue. However, during many surgical procedures, atelectasis (i.e., collapse of the lung) is induced to more easily resect the affected lung tissue. In many cases, especially during minimally invasive procedures, the surgeon may not be able to palpate the target tissue to identify the location of the lesion and/or fiducials. Therefore, the surgeon must rely solely upon intra-operative imaging to identify the implanted fiducials, which, as noted above, may be difficult when atelectasis is induced due to the increase in tissue density caused by the overlapping lung tissue.

Without being able to identify the lesion and/or fiducial, myriad complications may arise. Specifically, the surgeon may resect either too little or too much tissue, resulting in too little or too much margin. Additionally, if the surgeon is unable to remove the fiducial during the procedure, complications such pneumothorax, fiducial migration into pleural space, the airways, or into pulmonary lesions, lung neoplasm, etc., may arise. These and other complications necessitate further procedures to be performed, thereby increasing the probability of complications and increasing patient recovery time.

### SUMMARY

The present disclosure is directed to a surgical system including a fiducial, a surgical instrument, and an indicator. The fiducial is configured to insertion into target tissue and has an RFID tag disposed therein. The surgical instrument is capable of receiving information from the RFID tag when the surgical instrument is placed in proximity to the fiducial and is configured to perform a surgical function. The indicator is coupled to the surgical instrument and is configured to indicate when the surgical instrument is in proximity to the fiducial.

In a further aspect, the system may include a reader disposed within the surgical instrument that is configured to receive information from the RFID tag when the surgical instrument is placed in proximity to the fiducial.

In another aspect, the surgical function may be cutting tissue.

In yet another aspect, the surgical instrument may be configured for use in a minimally invasive surgical procedure.

In still another aspect, the surgical instrument may be a laparoscopic electrosurgical device.

In a further aspect, the RFID tag may be configured to store a unique identifier.

In another aspect, the system may further include a memory storing data relating to the RFID tag and one or more software applications.

In yet another aspect, the indicator may be a display coupled to a processor executing one of the one or more software applications to present the data relating to the RFID tag.

In still another aspect, the system may further include a user interface presented on the display in combination with one or more images of a patient stored within the memory. The user interface enables the identification of an image location depicting the fiducial within a patient's lungs.

In a further aspect, the user interface may be configured to present a location of a plurality of fiducials on one or more images of the patient stored within the memory.

A further aspect of the present disclosure is directed to a method of performing a surgical procedure including acquiring an image of a patient's lungs, identifying an area of interest on the acquired image, navigating an implantation tool to the area of interest that is capable of implanting a fiducial within target tissue, implanting a fiducial within target tissue, the fiducial including an RFID tag, navigating a surgical instrument to the area of interest, receiving information from the RFID tag using the surgical instrument, indicating that the surgical instrument is in proximity to the fiducial, and performing a surgical function on the target tissue using the surgical instrument.

In aspects, performing a surgical function on the target tissue may include using the surgical instrument to cut tissue.

In another aspect, receiving information from the RFID tag may include receiving information form the RFID tag using a reader disposed within the surgical instrument.

In a further aspect, the method may further include storing a unique identifier within a memory coupled to the RFID tag.

In yet another aspect, the method may further include storing data relating to the RFID tag in a memory coupled to a computer. The memory also stores one or more software applications.

In a further aspect, storing data relating to the RFID tag may include storing data relating to the location of the RFID tag within the target tissue.

In another aspect, storing data relating to the RFID tag may include storing data relating to the target tissue and the location of the RFID tag within a patient's lungs.

In yet another aspect, indicating that the surgical instrument is in proximity to the fiducial may include presenting the location of the RFID tag within the patient's lungs on a display coupled to a processor executing one of the one or more software applications.

In still another aspect, implanting a fiducial within target tissue may include implanting a plurality of fiducials at a plurality of locations within target tissue.

In another aspect, identifying an area of interest may include identifying a plurality of areas of interest on the acquired image and implanting a respective fiducial within target tissue within a respective area of interest of the plurality of areas of interest.

### BRIEF DESCRIPTION OF THE DRAWINGS

Various aspects and features of the present disclosure are described hereinbelow with references to the drawings, wherein:

FIG. 1 is a perspective view of a system provided in accordance with the present disclosure capable of navigating an implantation tool to an area of interest and implanting a fiducial within target tissue;

FIG. 2 is a cross-sectional view of a portion of a patient's lung showing a fiducial implanted within target tissue;

FIG. 3 is a cross-sectional view of a portion of a patient's lung showing a plurality of fiducials implanted within target tissue;

FIG. 4 is a cross-sectional view of a portion of a patient's lung showing a plurality of fiducials implanted within a corresponding plurality of target tissue;

FIG. 5 is a perspective view of an illustrative embodiment of a fiducial in accordance with the present disclosure;

FIG. 6 is a side view of a surgical instrument suitable for use with the system of FIG. 1; and

FIG. 7 is a cross-sectional view of a patient's lung showing the surgical instrument of FIG. 6 in proximity to a fiducial implanted within target tissue.

### DETAILED DESCRIPTION

The present disclosure is directed to communicating localization markers and methods of use thereof for more accurately identifying lesions within the lungs. As described herein, the localization markers may include a fiducial capable of emitting an RFID signal in response to an electromagnetic signal. The fiducials may be implanted within target tissue within the lungs of a patient using a navigational system. During a minimally invasive surgical procedure, a surgical instrument including a reader disposed therein is advanced within the lungs of the patient, and once in proximity to a fiducial that has been implanted within target tissue, indicates to a clinician that the surgical instrument is adjacent the target tissue. The navigation system may generate a 3D model of the lungs, on which the location of each fiducial that has been implanted may be illustrated. In this manner, a map of the implanted fiducials can be generated to aid a clinician during the surgical procedure. As can be appreciated, imaging modalities may be utilized to identify the location of the fiducials within the target tissue and to ensure that no fiducials remain within the tissue after completion of the surgical procedure. Similarly, a reader held external to the patient may be utilized to identify any fiducials remaining within the patient to eliminate the need for imaging the patient. The systems and methods of the present disclosure enable a clinician to more accurately identify the location of target tissue, enable more accurate surgical margins to be achieved, and mitigate the risk of any fiducials being left within the patient.

Embodiments of the present disclosure are now described in detail with reference to the drawings in which like reference numerals designate identical or corresponding elements in each of the several views. As used herein, the term “clinician” refers to a doctor, a nurse, or any other care provider and may include support personnel. Throughout this description, the term “proximal” will refer to the portion of the device or component thereof that is closer to the clinician and the term “distal” will refer to the portion of the device or component thereof that is farther from the clinician. Additionally, in the drawings and in the description that follows, terms such as front, rear, upper, lower, top, bottom, and similar directional terms are used simply for convenience of description and are not intended to limit the disclosure. In the following description, well-known functions or constructions are not described in detail to avoid obscuring the present disclosure in unnecessary detail.

Although the communicating localization markers and methods of use detailed herein are generally described with respect to the lungs, it is contemplated that the communicating localization markers and methods of use may be applied to any organ or tissue requiring treatment of an interior portion thereof (i.e., the liver, kidneys, or the like). As can be appreciated, it is envisioned that the surgical instruments detailed herein may be used during endoscopic, laparoscopic, or thoracoscopic approaches.

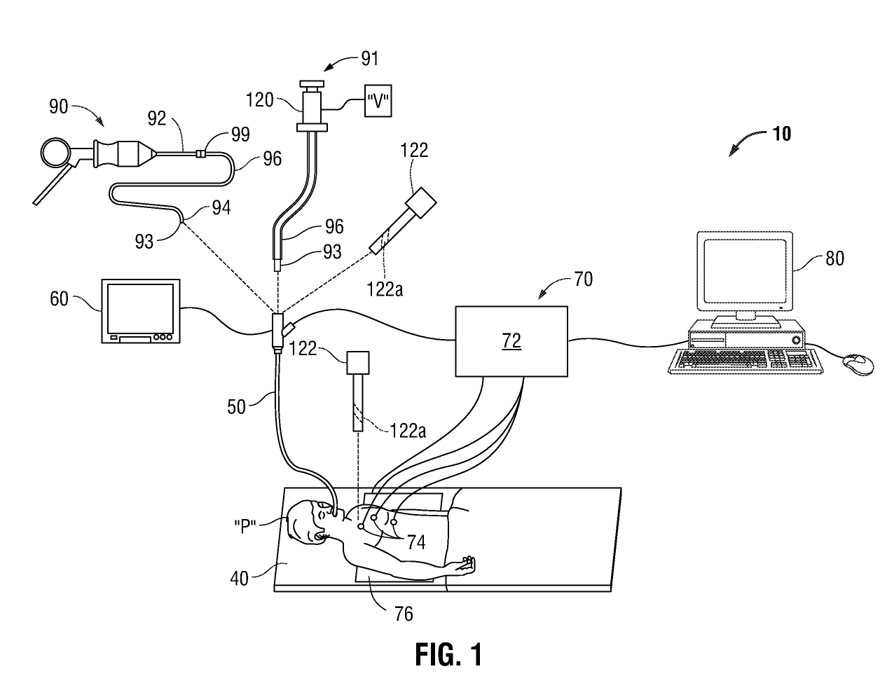

With reference to FIGS. 1-7, a method of performing surgery using communicating localization markers is described. Initially, a patient is imaged using any suitable imaging device (not shown), such as MRI, ultrasound, CT scan, Positron Emission Tomography (PET), metabolic scanning, or the like, and the images are stored within a memory coupled to a computer 80 (FIG. 1). The memory may include any non-transitory computer-readably storage media for storing data and/or software that is executable by a processor (not shown), e.g., solid-state, volatile, removable, and non-removable.

Following imaging, a software application may be initiated to enable review of the image data. One example of such an application is the ILOGIC® planning and navigation suites currently marketed by Medtronic. An area of interest illustrating the effects of lung disease (e.g., emphysema, COPD, asthma, cancer, or the like) is identified in the images and its location determined within the lungs “L” of the patient. Several methods of identifying an area of interest are contemplated such as ultrasound, CT scan, metabolic scanning, or the like. In one non-limiting embodiment, where the patient is not suffering from easily identified lesions or cancers of the lungs, the results of images generated from a CT scan can be analyzed to identify areas of hypodensity. Hypodense portions of the lungs are areas where the density of the tissue is less than the surrounding tissue. This may be particularly useful for patients suffering from emphysema as the expanded floppy alveoli or bullae will provide images that have areas which may be substantially darker or blacker than the surrounding tissue, indicating that they are largely air with little to no tissue separating these enlarged alveoli. Because of this hypodensity, image analysis using 3D image processing is particularly useful as identification of the areas where the densities of the images (measured in Hounsfield units or HU) is below a certain threshold (e.g., 950 HU) approximately the same as air. This 3D rendering is relatively straightforward and even coarse thresholding can be employed to distinguish the enlarged alveoli from tissue and identify their locations in the CT images. These coarse threshold values can then be rendered as a 3D model of the affected areas of the lungs. Techniques for generating 3D volumetric renderings are described in U.S. patent application Ser. No. 14/821,950 to Bharadwaj et al. entitled “Treatment Procedure Planning System and Method,” filed Aug. 10, 2015, the entire contents of which are incorporated by reference herein. In an alternative embodiment, PET imaging may be utilized to identify areas of low metabolic activity within the lungs. As can be appreciated, a device capable of performing a combined PET/CT imaging technique may be utilized, which has proven to be quite accurate. These areas of very little metabolic activity should closely correspond to areas of overinflated alveoli. There is very little metabolic activity in these areas because they are mostly comprised of air. In this way, a PET image set can be utilized to identify the hypodense areas to which navigation and treatment should be directed. After careful analysis, using one of the above described techniques, the location of the area of interest may be identified and its location stored within the memory coupled to the computer 80 (FIG. 1).

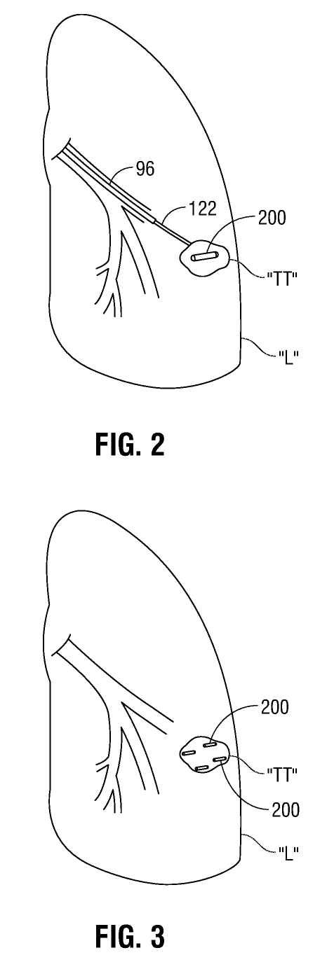

Next, the clinician utilizes the software to determine a pathway through which a mechanism or tool 122 for implanting a communicating localization marker or fiducial 200 (FIG. 2) within or adjacent the target tissue “TT” may be advanced within the patient using a percutaneous approach. As can be appreciated, the fiducial 200 may be implanted using any suitable tool 122 capable of being advanced within the thoracic cavity of a patient, either by penetrating tissue or being advanced through a trocar or other suitable device. It is contemplated that the tool 122 may be a fine aspiration needle, biopsy needle, catheter, or the like. In embodiments, it is contemplated that the fiducial 200 may be integrated within a medical device, such as a clamp, valve, stent, mesh, or any other medical device suitable for implantation within the lungs “L.” The progress of the tool 122 is monitored using any suitable intraoperative imaging modality, such as fluoroscopy, CT, or the like. Alternatively, it is envisioned that the progress of the tool 122 may be monitored using an electromagnetic navigation system. In this manner, a distal portion of the tool 122 may include a sensor 122a disposed thereon capable of being tracked using a transmitter mat 76, reference sensors 74, and a tracking module 72, as will be described in further detail hereinbelow.

In embodiments, the clinician may utilize the software to determine a pathway through the luminal network of the lungs “L” to the area of interest. In this manner, a bronchoscope 50 (FIG. 1) may be inserted within the patient and navigated through the patient's airways and adjacent the area of interested using a tracking system 70 (FIG. 1). However, if the bronchoscope 50 is unable to be navigated to the area of interest due to the size of the bronchoscope 50 prohibiting further insertion, a locatable guide (LG) 92 and an extended working channel (EWC) 96 (FIG. 1) may be advanced within a working channel of the bronchoscope 50 and independently navigated to the area of interest via tracking system 70. As can be appreciated, any suitable navigation and/or tracking system may be utilized to navigate the LG 92 and EWC 96 to the area of interest.

In embodiments, once each fiducial 200 is implanted within the target tissue, the patient may be imaged using any suitable imaging modality to identify the location of each fiducial 200 within the target tissue. In this manner, the distance to the outer edge of the lesion from the fiducial 200 may be determined to enable the clinician to more accurately determine how much tissue is required to be removed in order to obtain proper surgical margins. As can be appreciated, a plurality of fiducials 200 (FIG. 3) may be utilized to more accurately identify the amount of tissue to be removed to achieve the required surgical margins. In this manner, the clinician may implant individual fiducials 200 around the periphery of the target tissue “TT” at a location that would ensure proper surgical margin. Specifically, the fiducials 200 may be placed such that if after the clinician resects a portion of the target tissue “TT,” any or all of the implanted fiducials 200 remain, the clinician will recognize that not enough tissue has been resected to ensure the proper surgical margin. As can be appreciated, the clinician may be able to make several incisions during the same surgical procedure to ensure proper surgical margin, rather than having to undergo several individual surgical procedures. Similarly, by ensuring proper surgical margins repeat microwave treatments may be avoided, and in some instances ensure that drug therapies, such as chemotherapy, have been applied to the entire region to be treated. .

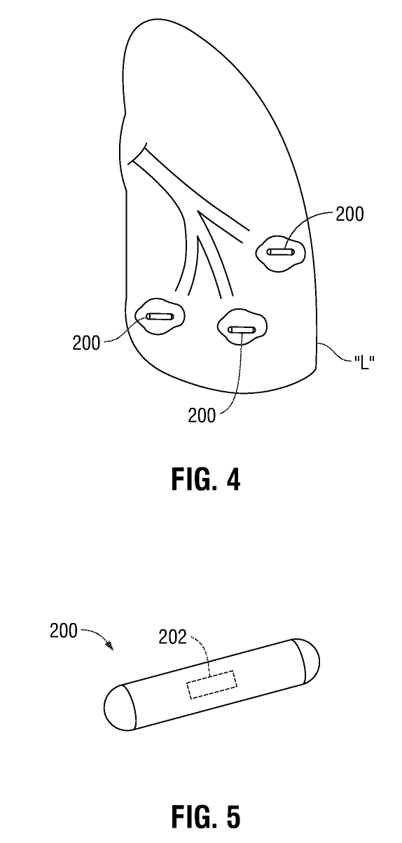

It is further contemplated that a plurality of fiducials 200 may be implanted in a corresponding plurality of lesions (FIG. 4) identified in a patient's lungs “L”. In this manner, each fiducial 200 may be correlated to each respective lesion in which it is implanted. Using the unique identification of each fiducial 200, a clinician can identify each specific lesion within the lungs “L” during the surgical procedure, as will be described in detail hereinbelow.

With reference to FIG. 5, the fiducial 200 includes a generally cylindrical configuration, although other configurations are also contemplated, such as hexagonal, square, oval, triangular, or the like. Although generally illustrated as having a smooth outer surface, it is contemplated that the fiducial 200 may include ridges, crenellations, prongs, or other similar means for inhibiting the fiducial 200 from migrating from its implanted position or to otherwise stabilize the fiducial 200 in the target tissue. The fiducial 200 may be formed from any suitable biocompatible material, such as glass, stainless steel, titanium, polymers (e.g., polyethelene, polyetheretherketone (PEEK), ethylene vinyl acetate, polyphenylsulfone (PPSU), polysulfone (PSU)), cobalt chrome, composites, or the like. It is contemplated that the fiducial 200 may be formed from a radio opaque or, alternatively, a radiolucent material and may be formed from a material that is MRI compatible or MR safe. Although generally described as including a Radio Frequency Identification (RFID) tag 202 disposed therein for identification, as will be described in further detail hereinbelow, it is contemplated that the fiducial 200 may be formed from various other materials that will enable a clinician to identify the location of the fiducial 200 within the patient should the RFID tag 202 fail or otherwise not respond to the reader 308 (described in further detail hereinbelow). In this manner, a clinician may utilize imaging modalities to identify a fiducial 200 formed from a radiolucent material. It is further contemplated that the fiducial 200 may be formed from a metallic material such that the fiducial 200 may be detected using any suitable metal detection technology known in the art, such as Very Low Frequency (VLF), Pulse Induction (PI), or Beat-frequency Oscillation (BFO). In embodiments, the fiducial 200 may include coils, antennas, inductive-capacitance (LC) or resistive-inductive-capacitive (RLC) circuits that may be configured to resonate at a specific frequency to aid in identification of the fiducial 200. These and other modalities known in the art enable a clinician to identify the location of the fiducial 200 within the lungs “L,” thereby enabling a clinician to remove the fiducial 200 from the patient to avoid complications arising from a fiducial left in tissue and to avoid having to perform additional surgical procedures to ensure each fiducial has been removed.

The fiducial 200 includes an RFID tag 202 disposed in an interior portion thereof. It is contemplated that the RFID tag 202 may be any suitable RFID tag known in the art and may have a factory assigned serial number or may be field programmable (write-once, read-multiple, etc.). In one non-limiting embodiment, the RFID tag 202 is a passive tag, although it is contemplated that the RFID tag 202 may be an active RFID tag. In this manner, the fiducial 200 may include an active RFID tag 202 having an internal power storage device (not shown), such as a capacitor, battery, or the like. Examples of an active RFID tag utilizing an internal power storage device other than a battery are active RFID tags marketed and sold by Tagent. As can be appreciated, the frequency at which the RFID tag 202 operates depends on the needs of the procedure being performed. However, it is contemplated that the RFID tag 202 may operate at Low Frequency (125-134 KHz), High Frequency and Near-Field Communication (13.56 MHz), or Ultra High Frequency (865-960 MHz).

The RFID 202 tag is configured to store information pertaining to the location of the fiducial 200 within the patient. In this manner, the RFID tag 202 may be programmed to identify the target tissue where the fiducial 200 has been implanted (in a case where multiple areas of interest have been identified), the location of the fiducial 200 within the target tissue “TT” (i.e., in the center, on the periphery, on the margin, etc.), or any other identifying characteristics of the tissue or the location at which the fiducial 200 has been implanted.

It is contemplated that each RFID tag 202 may have a unique factory identified serial number (i.e., not field programmable). In this manner, as each fiducial 200 is implanted within the target tissue, the serial number associated with the RFID tag 202 disposed within each fiducial 200 is recorded by the clinician (manually or entered into the computer 80). Once each fiducial 200 is implanted and the RFID tag 202 associated with the specific area of interest in which the fiducial 200 is implanted, it is envisioned that the 3D model generated during the planning procedure (described above) can display the location of each fiducial 200, thereby creating a map of each fiducial 200 within the 3D model. It is contemplated that as a surgical instrument, to be described in greater detail below, is placed in proximity to a fiducial 200, the map can identify which fiducial 200 the surgical instrument is near using any suitable means, such as illumination, a pop-up notification, or the like. Alternatively, the map may be generated and integrated with or overlaid on a previously acquired image (via any of the above noted imaging modalities) of the patient's lungs “L” and displayed on any suitable device, and in one non-limiting embodiment, may be displayed on a display monitor associated with the computer 80 or the monitoring equipment 60 (FIG. 1).

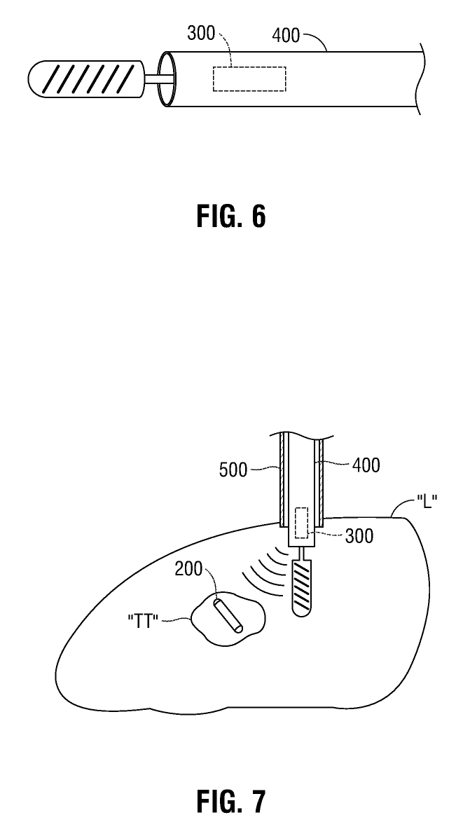

An RFID interrogator or reader 300 (FIG. 6) is used to identify the location of the fiducial 200 implanted within the target tissue. As can be appreciated, the type of reader 300 utilized depends on the type of RFID tag 202 employed. In the case where the RFID tag 202 is a passive tag, the reader 300 may be an Active Reader Passive Tag (ARPT) system, where the reader 300 is capable of transmitting interrogation signals and receiving corresponding responses from the RFID tag 202. Alternatively, in the case where the RFID tag 202 is an active tag, the reader 300 may be a Passive Reader Active Tag (PRAT), where the reader 300 is configured to receive radio signals from the RFID tag 202, or an Active Reader Active Tag (ARAT). In the case where the RFID tag 202 is an ARAT, the RFID tag 202 is initially in a sleep state where the RFID tag 202 does not emit any signal. When the clinician wants to read the RFID tag 202, the reader 300 sends an interrogation signal to awaken the RFID tag 202, at which point the RFID tag 202 emits a signal that is received by the reader 300. It is contemplated that the reader 300 may be toggled using a button (not shown) or other device requiring input from the clinician or may constantly emit an interrogation signal as the reader is powered. It is further envisioned that one or more of the above noted types of readers 300 and RFID tags 202 may be employed, depending upon the needs of the surgical procedure being performed.

The reader 300 is configured to be coupled to various surgical instruments such that the fiducial 200 may be identified during the surgical procedure without the need for additional probes requiring the formation of additional incisions, continued removal or insertion of a probe through a trocar or EWC, and eliminates the need for intra-operative imaging modalities to identify the location of the fiducials 200 implanted within the target tissue. In this manner, the reader 300 is disposed within a portion of a surgical instrument (FIG. 6) capable of performing a surgical function, such as cutting, coagulating, stapling, ablating, applying clips, taking a biopsy, or any other surgical procedure known in the art. It is envisioned that the surgical instrument may be any surgical instrument capable of being used during a surgical procedure, such as electrosurgical devices, staplers, clip appliers, biopsy devices, microwave ablation devices, or any suitable surgical device currently marketed and sold by Medtronic. As can be appreciated, the surgical instrument may be capable of being used in minimally invasive procedure or an open surgical procedure.

In one non-limiting embodiment, the reader 300 is disposed within a laparoscopic electrosurgical instrument 400, such as laparoscopic electrodes marketed and sold by Medtronic. The reader 300 is disposed at a distal portion of the electrosurgical instrument 400 (FIG. 6) such that as the distal end of the electrosurgical instrument 400 is placed in proximity to the fiducial 200 (FIG. 7), the reader 300 can interrogate the RFID tag 202 and provide an indication to the clinician that the electrosurgical instrument 400 is in proximity to the fiducial 200, and in some instances, identify the fiducial 200, and therefore the lesion, in which the electrosurgical instrument 400 is in proximity to. In embodiments, the indication can be through a display monitor (not shown) associated with the computer 80 or the monitoring equipment 60 (FIG. 1), or through the electrosurgical instrument 400 itself. In this manner, the reader can provide audio/visual or haptic feedback to alert the clinician that the electrosurgical instrument 400 is in proximity to a fiducial 200.

With reference to FIG. 7, in the case where only one fiducial 200 has been implanted within the area of interest, the clinician advances the electrosurgical instrument 400 within the trocar 500 or other suitable device until the reader 300 identifies an RFID tag 202. At this point, the clinician has identified the location of the fiducial 200 within the area of interest and has determined the amount of tissue surrounding the fiducial 200 that is required to be removed in order to obtain the proper surgical margin. It is contemplated that as the tissue is dissected, the clinician may inject dye or another suitable medium to identify the edges of the incision to aid in excising the target tissue “TT.”

Alternatively, in the case where a plurality of fiducials 200 have been implanted around the periphery or within the area of interest, the clinician has previously identified the location of each fiducial 200 in relation to the area of interest and has determined the amount of tissue surrounding the fiducial 200 that is required to be removed in order to obtain the proper surgical margin. In this manner, the use of a plurality of fiducials 200 enables a clinician to obtain a finer margin (i.e., minimal amount of excess tissue surrounding the lesion) by placing the electrosurgical instrument 400 adjacent each fiducial while dissecting the tissue. It is contemplated that the clinician may dissect the tissue while sequentially following each fiducial 200 implanted within the tissue. Guidance, such as in the form of pathway planning, may be utilized by illustrating the preferred path to each fiducial 200, either on a previously acquired image, or in the 3D model generated during the pathway planning procedure described above with respect to the navigation system described in detail hereinabove.

It is contemplated that once the clinician has excised the target tissue, a device (not shown) including a reader 300 and capable of reading the RFID tags 202 from a position external to the patient may be utilized to determine if there are any remaining fiducials 200 within the patient. As can be appreciated, the device may be coupled to the computer 80 or monitoring equipment 60 (FIG. 1) such that any remaining fiducials 200 that are detected may be displayed on the map or 3D model. If any fiducials 200 remain, the procedure described above may be performed as many times as necessary to ensure that all of the fiducials 200 have been removed from the patient. In this manner, the remaining fiducials 200 may be identified intra-operatively to avoid having to perform subsequent procedures, thereby decreasing pain, recovery time, potential for complications, and the like.

Alternatively, once the procedure has been completed, the patient may be imaged using any suitable imaging modality, such as those detailed hereinabove, to identify any remaining fiducials 200. If any fiducials 200 remain, the procedure described above may be performed as many times as necessary to ensure that all of the fiducials 200 have been removed from the patient.

Referring again to FIG. 1, a system 10 including a navigation system capable of guiding the bronchoscope 50 through the luminal network, or the tool 122 via a percutaneous approach, to an area of interest is illustrated. Patient “P” is shown lying on operating table 40 with bronchoscope 50 inserted through the patient's mouth and into the patient's airways. Bronchoscope 50 includes a source of illumination and a video imaging system (not explicitly shown) and is coupled to monitoring equipment 60, e.g., a video display, for displaying the video images received from the video imaging system of bronchoscope 50. In embodiments, it is contemplated that bronchoscope 50 may be any suitable bronchoscope capable of navigating the airways of a patient and permitting a suitable tool 122 (such as a tool capable of implanting a fiducial 200 or a biopsy device) to be inserted therein. For a detailed description of an exemplary bronchoscope 50, reference can be made to U.S. Patent Application Publication No. 2015/0265257 to Costello et al. entitled “Systems, and Methods for Navigating a Biopsy Tool to a Target Location and Obtaining a Tissue Sample Using the Same”, filed Dec. 9, 2014, the entire contents of which are incorporated by reference herein.

The navigation system may be a six degrees-of-freedom electromagnetic tracking system 70, e.g., similar to those disclosed in U.S. patent application Ser. No. 14/753,288 to Brown et al. entitled “System and Method for Navigating within the Lung”, filed Jun. 29, 2015 and published PCT Application Nos. WO 00/10456 and WO 01/67035, the entire contents of each of which is incorporated herein by reference, or other suitable positioning measuring system, is utilized for performing registration and navigation, although other configurations are also contemplated. Tracking system 70 includes a tracking module 72, a plurality of reference sensors 74, and a transmitter mat 76. Tracking system 70 is configured for use with either positioning assembly 90 or positioning assembly 91, a tool 122 capable of implanting a fiducial 200, or any suitable biopsy device, as detailed below. Positioning assemblies 90 and 91 further include EWC 96 and a handle 120. LG 92 and EWC 96 are configured for insertion through a working channel of bronchoscope 50 into the patient's airways (although LG 92 and EWC 96 may alternatively be used without bronchoscope 50) and are selectively lockable relative to one another via a locking mechanism 99. Distal tip 93 of LG 92 may be configured for steering in any suitable fashion, e.g., using a plurality of steering wires (not shown) coupled between handle 98 and distal tip 93, to facilitate maneuvering distal tip 93 of LG 92 and EWC 96 through the patient's airways. Alternatively, rotation and translation of handle 120 may facilitate maneuvering of the distal tip 93 of LG 92, and in particular embodiments the EWC 96 may be angled or curved to assist in maneuvering the distal tip 93 through the airways. Sensor 94 is integrated with distal tip 93 of LG 92 and allows monitoring of the position and orientation of distal tip 93, in six degrees of freedom, relative to the reference coordinate system. For a detailed description of the construction of exemplary navigation systems, reference may be made to U.S. Patent Application Publication No. 2015/0265257 to Costello et al., previously incorporated by reference.

A transmitter mat 76 is positioned beneath the patient “P” and is a transmitter of electromagnetic radiation. Transmitter mat 76 includes a stack of three substantially planar rectangular loop antennas (not shown) configured to be connected to drive circuitry (not shown). For a detailed description of the construction of exemplary transmitter mats, which may also be referred to as location boards, reference may be made to U.S. Patent Application Publication No. 2009/0284255 to Zur entitled “Magnetic Interference Detection System and Method”, filed Apr. 2, 2009, the entire contents of which are incorporated by reference herein.

Transmitter mat 76 and the plurality of reference sensors 74 are interconnected with tracking module 72, which derives the location of each sensor 74 in six degrees of freedom. One or more of reference sensors 74 are attached to the chest of the patient “P.” The six degrees of freedom coordinates of reference sensors 74 are sent to computer 80 (which includes the appropriate software) where they are used to calculate a patient coordinate frame of reference. Registration, as detailed below, is generally performed by identifying locations in both the three-dimensional model and the patient's airways and measuring the coordinates in both systems. Further details of such a registration technique can be found in U.S. Patent Application Pub. No. 2011/0085720 to Barak et al. entitled “Automatic Registration Technique”, filed May 14, 2010, the entire contents of which are incorporated herein by reference, although other suitable registration techniques are also contemplated.

In use, with respect to the navigation phase, LG 92 is inserted into positioning assembly 90, 91, and EWC 96 such that sensor 94 projects from the distal end of EWC 96. LG 92 and EWC 96 are then locked together via locking mechanism 99 (for example). LG 92, together with EWC 96, are then inserted through bronchoscope 50 and into the airways of the patient “P,” with LG 92 and EWC 96 moving in concert with one another through bronchoscope 50 and into the airways of the patient “P.” Automatic registration is performed by moving LG 92 through the airways of the patient “P.” More specifically, data pertaining to locations of sensor 94 while LG 92 is moving through the airways is recorded using transmitter mat 76, reference sensors 74, and tracking module 72. A shape resulting from this location data is compared to an interior geometry of passages of the three-dimensional model generated in the planning phase, and a location correlation between the shape and the three-dimensional model based on the comparison is determined, e.g., utilizing the software on computer 80. In addition, the software identifies non-tissue space (e.g., air filled cavities) in the three-dimensional model. The software aligns, or registers, an image representing a location of sensor 94 of LG 92 with an image of the three-dimensional model based on the recorded location data and an assumption that LG 92 remains located in non-tissue space in the patient's airways. This completes the registration portion of the navigation phase. Similarly, advancing the tool 122 within the thoracic cavity of the patient using a percutaneous approach may be monitored. In this manner, the tool 122 may include a sensor 122a disposed on a distal portion thereof. Sensor 122a is similar to sensor 94 and may be tracked using the transmitter mat 76, the reference sensors 74, and the tracking module 72 and its location displayed on the three-dimensional model in a similar manner to sensor 94.

Referring still to FIG. 1, once the planning phase has been completed, e.g., the target tissue “TT” has been identified and the pathway thereto selected, and registration has been completed, system 10 may be utilized to navigate LG 92 through the patient's airway to the area of interest. To facilitate such navigation, computer 80, monitoring equipment 60, and/or any other suitable display may be configured to display the 3D model including the selected pathway from the current location of sensor 94 of LG 92 to the area of interest. Navigation of LG 92 to the area of interest using tracking system 70 is similar to that detailed above and thus, is not detailed here for the purposes of brevity.

Once LG 92 has been successfully navigated to the area of interest, completing the navigation phase, LG 92 may be unlocked from EWC 96 and removed, leaving EWC 96 in place as a guide channel for guiding a suitable tool 122 to implant a fiducial 200 within the target tissue. For a detailed description of exemplary navigation and planning phases, reference may be made to U.S. patent application Ser. No. 14/753,288 to Brown et al. , previously incorporated by reference.

The electromagnetic waves generated by transmitter mat 76 are received by the various sensor elements configured for use with the implantation tool or sensor 94 of LG 92, and are converted into electrical signals that are sensed via reference sensors 74. Tracking system 70 further includes reception circuitry (not shown) that has appropriate amplifiers and A/D converters that are utilized to receive the electrical signals from reference sensors 74 and process these signals to determine and record location data of the sensor assembly. Computer 80 may be configured to receive the location data from tracking system 70 and display the current location of the sensor assembly on the 3D model and relative to the selected pathway generated during the planning phase, e.g., on computer 80, monitoring equipment 60, or other suitable display. Thus, navigation of the implantation tool 122 and/or LG 92 to the target tissue “TT” and/or manipulation of the implantation tool 122 relative to the target tissue “TT,” as detailed above, can be readily achieved.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments.

What is claimed is:

1. A surgical system, comprising:
a fiducial configured for insertion into target tissue, the fiducial having an RFID tag disposed therein;
a surgical instrument capable of receiving information from the RFID tag when the surgical instrument is placed in proximity to the fiducial, the surgical instrument being configured to perform a surgical function; and
an indicator coupled to the surgical instrument, the indicator configured to indicate when the surgical instrument is in proximity to the fiducial.

2. The system of claim 1, further including a reader disposed within the surgical instrument, the reader configured to receive information from the RFID tag when the surgical instrument is placed in proximity to the fiducial.

3. The system of claim 1, wherein the surgical function is cutting tissue.

4. The system of claim 1, wherein the surgical instrument is configured for use in a minimally invasive surgical procedure.

5. The system of claim 4, wherein the surgical instrument is a laparoscopic electrosurgical instrument.

6. The system of claim 1, wherein the RFID tag is configured to store a unique identifier.

7. The system of claim 6, further including a memory storing data relating to the RFID tag and one or more software applications.

8. The system of claim 7, wherein the indicator is a display coupled to a processor executing one of the one or more software applications to present the data relating to the RFID tag.

9. The system of claim 8, further including a user interface presented on the display in combination with one or more images of a patient stored within the memory, the user interface enabling the identification of an image location depicting the fiducial within a patient's lungs.

10. The system of claim 9, wherein the user interface is configured to present a location of a plurality of fiducials on one or more images of the patient stored within the memory.

11. A method of performing a surgical procedure, comprising:
acquiring an image of a patient's lungs;
identifying an area of interest on the acquired image;
navigating an implantation tool to the area of interest, the implantation tool capable of implanting a fiducial within target tissue;
implanting a fiducial within target tissue, the fiducial including an RFID tag;
navigating a surgical instrument to the area of interest;
receiving information from the RFID tag using the surgical instrument;
indicating that the surgical instrument is in proximity to the fiducial; and
performing a surgical function on the target tissue using the surgical instrument.

12. The method of claim 11, wherein performing a surgical function on the target tissue includes using the surgical instrument to cut tissue.

13. The method of claim 12, wherein receiving information from the RFID tag includes receiving information from the RFID tag using a reader disposed within the surgical instrument.

14. The method of claim 11, further including storing a unique identifier within a memory coupled to the RFID tag.

15. The method of claim 14, further including storing data relating to the RFID tag in a memory coupled to a computer, the memory storing one or more software applications.

16. The method of claim 15, wherein storing data relating to the RFID tag includes storing data relating to the location of the RFID tag within the target tissue.

17. The method of claim 15, wherein storing data relating to the RFID tag includes storing data relating to the target tissue and the location of the RFID tag within a patient's lungs.

18. The method of claim 16, wherein indicating that the surgical instrument is in proximity to the fiducial includes presenting the location of the RFID tag within the patient's lungs on a display coupled to a processor executing one of the one or more software applications.

19. The method of claim 11, wherein implanting a fiducial within target tissue includes implanting a plurality of fiducials at a plurality of locations within target tissue.

20. The method of claim 11, wherein identifying an area of interest includes identifying a plurality of areas of interest on the acquired image and implanting a respective fiducial within target tissue within a respective area of interest of the plurality of areas of interest.
